(19) United States
(12) Patent Application Publication
(10) Pub. No.: US 2022/0296533 A1
(43) Pub. Date: Sep. 22, 2022

(54) COMPOSITIONS AND METHODS FOR INCREASING INTRACELLULAR GLUCOSE UPTAKE

(71) Applicant: RUSS IP HOLDING GROUP LLC, Houston, TX (US)

(72) Inventor: Kenneth O. Russell, Houston, TX (US)

(21) Appl. No.: 17/570,296

(22) Filed: Jan. 6, 2022

Related U.S. Application Data
(63) Continuation-in-part of application No. 17/207,979, filed on Mar. 22, 2021.
Application No. 17/207,979 is a continuation-in-part of application No. 17/320,212, filed on May 13, 2021.

(51) Int. Cl. A61K 31/047; A61K 33/24; A61K 47/55

(52) U.S. Cl. CPC A61K 31/047; A61K 33/24; A61K 47/551

(57) ABSTRACT

A method for increasing intracellular glucose uptake by administering a composition comprising at least one Glut-4 expression inducement agent in combination with an antioxidant, an amino acid, and a vasodilator.

### CROSS-REFERENCE TO RELATED APPLICATION

This application claim the priority of a provisional application No. 63,253,490 filed on Oct. 7, 2021, and is a continuation-in-part of U.S. patent application No. 17,207,979 filed on 03-22-2021, which is a continuation of U.S. patent application No. 17,187,193 filed on Feb. 26, 2021, which is a reissue of U.S. patent application No. 12,069,505 filed on Feb. 11, 2008, now U.S. Pat. No. 9,585,898; a continuation-in-part of U.S. patent application No. 17,320,212 filed on May 13, 2021, which is a continuation-in-part of U.S. patent application No. 17,87,193 filed on Feb. 26, 2021 and U.S. patent application No. 16,442,512, which is a continuation-in-part of U.S. patent application No. 15,925,437, filed on Mar. 19, 2018, now U.S. Pat. No. 10,398,643, which is a continuation-in-part of U.S. patent application No. 15,154,671, filed on May 13, 2016, now U.S. Pat. No. 10,172,847. The entire contents of each of these applications is herein incorporated by reference for al purposes.

### BACKGROUND OF THE DISCLOSURE

Glucose is an important fuel for contracting muscle, and normal glucose metabolism is vital for health. With prolonged exercise, as the liver becomes depleted of glycogen and gluconeogenesis is unable to fully compensate, liver glucose output is reduced and hypoglycemia can limit muscle glucose uptake (Ahlborg G. et al, 1971; Felig P. et al, 1982). In contrast, increasing arterial glucose availability, by ingestion of carbohydrate-containing beverages, results in increased muscle glucose uptake and oxidation during prolonged exercise (Ahlborg G. et al, 1900; Jeukendrup A E et al, 1999; McConell G et al, 1994).

GLUT4 is a membrane-inserted protein that continuously cycles to and from the plasma membrane, undergoing a series of sorting steps in endomembranes (Dugani & Klip, 2005). It is one of 13 facilitative glucose transport proteins encoded in the genome and is expressed most abundantly in adipose tissue and cardiac and skeletal muscle. Thus, it appears that the skeletal muscle GLUT4 level correlates with the capacity for glucose uptake during very intense exercise, a finding consistent with the relationship between muscle GLUT4 content and glucose transport during intense electrical stimulation of skeletal muscles (Henriksen E J et al, 1990) and the relationship between GLUT4 expression and insulin action in skeletal muscle. In this regard, increased skeletal muscle GLUT4 expression would also facilitate postexercise glucose uptake and glycogen storage (Greiwe J S et al, 1999; McCoy M et al, 1996). studies have suggested that GLUT4 intrinsic activity can indeed be altered (Antonescu C N et al, 2011; Zaid H et al, 2009).

Insulin has been shown to increase GLUT4 abundance in an enriched muscle plasma membrane fraction (Goodyear U I et al, 1996; Guma A et al, 1995), and increased GLUT4 surface membrane content evaluated by surface labeling (MacLean D A et al, 1999) has been demonstrated after insulin stimulation.

In vivo study showed that erythritol increases glucose uptake in muscle cell with or without insulin, which suggests that erythritol may potentiate insulin-mediated glucose uptake in muscles. The study data suggested that erythritol may exert antihyperglycemic effects not only via reducing small intestinal glucose absorption, but also by increasing muscle glucose uptake, improving glucose metabolic enzymes activity and modulating muscle GLUT-4 and IRS-1 mRNA and protein expression (Chika I. C. et al, 2017). Thus, erythritol may be a useful dietary supplement for managing hyperglycemia in diabetic individuals.

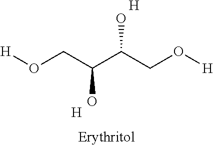

Caffeine causes release of Ca2+ from the sarcoplasmic reticulum, and it also causes an increase in glucose transport. Early studies showed that the increase in muscle glucose uptake during contractions does not require membrane depolarization but only release of Ca2+ (Holloszy et al, 1965; Holloszy J O et al, 1967). The study suggests that increase in Ca2+ per se is unlikely to increase muscle glucose uptake but that the effects of caffeine are due to the subsequent energy stress of the muscle which via activation of AMPK causes increased glucose uptake.

While membrane fluidity increased with increasing temperature, chromium picolinate or chromium nicotinate, pyridine carboxylate isomer complexes, significantly increase the fluidity when adding to the medium used for preparation of the liposomes (Evans G, Bowman T., 1992).

Chromium picolinate, produced the largest increase in membrane fluidity and also resulted in a marked increase in insulin internalization in cultured cells. It may affect the action of insulin through an effect on the rate of insulin internalization, which, by an unidentified mechanism, regulates the synthesis and/or insertion of insulin receptors into the plasma membrane. Because of the lipophilic nature of chromium picolinate, the complex may affect insulin internalization by maintaining a level of membrane fluidity necessary for efficient removal of the insulin-receptor complex from the surface of the plasma membrane.

The intestinal absorption of dietary chromium at daily intakes of 40 μg and more is approximately 0.5% of the total amount present. However, intakes of less than 40 μg/day are absorbed with an increasing efficiency, up to about 2% of the total (Anderson and Kozlovsky, 1985).

U.S. Pat. No. 5,948,772 discloses that the combination of chromium and nicotinic/picolinic acid, in effective amount between 50 μg and about 10,000 μg, facilitates the absorption of monovalent, divalent and trivalent metal ions by transporting them across intestinal cells and into the bloodstream.

Pat. App. No. WO2008126088A2 discloses that the doses of nicotinic acid over 50 mg may cause flushing of the skin, lasting about 60 minutes, along with a mild itching sensation and a reddening of the skin. Nicotinic acid can cause vasodilation of cutaneous blood vessels resulting in increased blood flow, principally in the face, neck and chest. This produces the niacin or nicotinic acid-flush. The niacin-flush is thought to be mediated via the prostaglandin (PG) prostacyclin and via histamine release. When the nicotinic acid is given repeatedly, tolerance to nicotinic acid-induced flushing tolerance within about a week.

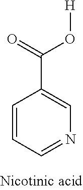

Canadian Pat. App. Mo. 2841731 discloses that nicotinamide (IUPAC name pyridine-3-carboxamide), also known as niacinamide and nicotinic acid amide, is the amide of nicotinic acid (vitamin B3/niacin). Nicotinamide is a water-soluble vitamin and is part of the vitamin B group. Nicotinic acid, also known as niacin, is converted to nicotinamide in vivo, and, though the two are identical in their vitamin functions, nicotinamide does not have the same pharmacologic and toxic effects of niacin, which occur incidental to niacin's conversion. Thus, nicotinamide does not reduce cholesterol or cause flushing. Additionally, nicotinamide lacks of the vasodilator, gastrointestinal, hepatic, and hypolipidemic actions of nicotinic acid. As such, nicotinamide has not been shown to produce the flushing, itching, and burning sensations of the skin as is commonly seen when large doses of nicotinic acid are administered orally.

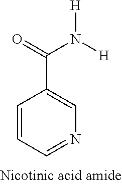

Studies have shown that providing additional supplementation of 3-10 g arginine (IUPAC name: 2-amino-5-(diaminomethylidene amino) pentanoic acid) three times daily potentially increase vasodilation, circulation, delivery of NAD precursor, and angiogenesis (Penberthy, W. T., 2012). Arginine is well known to promote vasodilation during exercise (Kubota T. et al, 1997) or hypercholestermia. Niacin increases brain endothelialnitric oxide synthase protein expression which is known to cause increased basal vasodilation and angiogenesis (Chen J. et al., 2007). Thus, it would be reasonable to include additional arginine to sustain the vasodilation.

Arginine is also the main biological precursor of nitric oxide (NO) and has been described to improve insulin sensitivity in diabetes and obesity (Thais C. B et al, 2013). Arg is a natural and exclusive physiological substrate of nitric oxide synthase (NOS) and consequently, it is the main biological precursor of nitric oxide (NO) (Palmer R M J et al, 1988). NO is a key metabolic and vascular regulator, as well as an endogenous messenger molecule involved in the regulation of cell metabolism, insulin signaling and secretion, neurotransmission and immune system function (Newsholme P et al, 2010). Three NOS isoforms have been identified for catalyzing the oxidation of arginine to NO and L-citrulline (Iyengar R et al, 1987). They are classified as isoform I, in neuronal (nNOS) and epithelial cells; isoform II, in cytokine-induced cells (inducible NOS, iNOS) and isoform III, in endothelial cells (eNOS) (Iyengar R et al, 1987; Forstermann U et al, 1994).

Arginine treatment has a dual effect to directly enhance skeletal muscle glycogen synthesis and lipid oxidation, and indirectly potentiate insulin-stimulated glucose uptake (Thais C. B et al, 2013). Administration of arginine improves insulin sensitivity in obese individuals and type 2 diabetic patients (Wascher T C et al, 1997) by increasing Akt phosphorylation and GLUT4 protein content in skeletal muscle cells, without altering IRS1 Tyr612 phosphorylation. The increase in GLUT4 protein abundance was associated with a trend for increased plasma membrane GLUT4 content, which may account for improvements in glucose transport. Arginine treatment also enhanced basal GSK-3α/β phosphorylation and glycogen synthesis. These metabolic and signaling responses are a direct effect of arginine, rather than a secondary effect due to changes in muscle differentiation, since protein abundance of several differentiation markers was unaltered. Thus, arginine treatment improves glucose metabolism, both via an acute effect to enhance signal transduction and a genomic effect to increase protein abundance.

Chronic arginine treatment increases glucose uptake and metabolism, as well as palmitate oxidation in L6 skeletal muscle cells. These metabolic changes are coincident with increased NO/c-GMP levels and signal transduction via Akt and AMPK. Inhibition of nitric oxide synthase by L-NAME abolished the effects of arginine. These findings indicate that arginine improves glucose and lipid metabolism in skeletal muscle via the NO/c-GMP cascade. Taken together with earlier studies highlighting a role for NO in glucose metabolism in skeletal muscle (Morita M. et al, 2013; Kameda N. et al, 2011; Pollock J. S. et al, 1991; Shin S. et al, 2011) and the evidence implicating arginine as the exclusive biological precursor of NO, our findings suggest that the amino acid arginine holds promise as a safe and cost effective nutrient (Romero M. J. et al, 2006) that may improve the metabolic profile in skeletal muscle in obesity and type 2 diabetes. Additional studies are required to evaluate the dynamic state of arginine metabolism in vivo, since this amino acid also increases GH secretion, which has a potent diabetogenic effect that may have a deleterious impact on skeletal muscle insulin sensitivity.

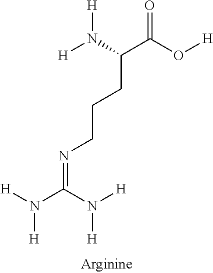 [SMILES: NC(N)=NCCC[C@H](N)C(=O)O]

Citrulline (IUPAC name: (2S)-2-amino-5-(carbamoylamino) pentanoic acid) is generally recognized as safe for oral use (Chen P Y et al, 1971). In contrast to arginine or ornithine, which induce gastrointestinal side effects at high doses (i.e., >10 g in one bolus) (Preli R B et al., 2002), citrulline is well tolerated. This could be explained by the rapid saturation of the intestinal absorption of arginine and ornithine inducing osmotic diarrhea at high loads. This difference between these amino acids suggests that the intestinal absorption of citrulline is not a limiting step in citrulline bioavailability, even at high citrulline loads.

Citrulline possesses a highly specific metabolism that bypasses splanchnic extraction because it is not used by the intestine or taken up by the liver. The administration of citrulline may be used to deliver available nitrogen for protein homeostasis in peripheral tissues and as an arginine precursor synthesized de novo in the kidneys and endothelial and immune cells in some cells, arginine can be recycled from citrulline, which is of major importance in the so-called nitric oxide (NO) cycle. Hence, citrulline can act as an arginine precursor for NO synthesis, and it plays an important role in NO metabolism and regulation. Citrulline administration therefore may offer a therapeutic strategy for controlling NO metabolism disorders and improving cardiovascular function. Citrulline supplementation should be considered for use in all circumstances or disease states in which arginine has beneficial effects and/or where arginine supplementation may be considered harmful. Furthermore, aging is associated with a high splanchnic sequestration of amino acids and sarcopenia, so citrulline, which escapes splanchnic extraction and can stimulate muscle protein synthesis, could be a valuable tool for delivering adequate amounts of nitrogen to peripheral tissues, including the muscle, in elderly subjects.

L-Citrulline plus L-arginine supplementation caused a more rapid increase in plasma L-arginine levels and marked enhancement of NO bioavailability, including plasma cGMP concentrations, than with dosage with the single amino acids. Blood flow in the central ear artery in rabbits was also significantly increased by L-citrulline plus L-arginine administration as compared with the control. The combination of combination of oral L-citrulline and L-arginine effectively and rapidly augments NO-dependent responses at the acute stage.

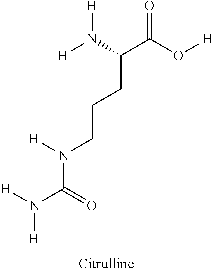 [SMILES: NC(=O)NCCC[C@H](N)C(=O)O]

Recent studies who that herb such as Nigella sativa (N. sativa) improves glucose homeostasis and serum lipids in type 2 diabetes. Clinical and statistical significant reduction in FBS and HbA1c levels following N. sativa consumption provides strong evidence for incorporation of N. sativa as part of therapy in diabetes (Daryabeygi-Khotbehsara R., 2017). Mechanisms underlying the therapeutic effects of N. sativa on glycemia suggest amelioration of pancreatic β-cells leading to insulin secretion (Alimohammadi S. et al, 2013 and Kanter M. et al, 2003) reducing hepatic gluconeogenesis (Fararh K. et al, 2004) and inducing insulin sensitivity in peripheral tissue (Benhaddou-Andaloussi A et al, 2004). These effects are attributable to active ingredients of N. sativa (e.g. thymoquinone, dithymoquinone, linoleic acid and oleic acid). Study has shown that thymoquinone treatment on streptozotocin-nicotinamide-induced diabetic rats exerted hypoglycemic benefits (Pari L. et al, 2009). The administration of thymoquinone significantly lowers plasma blood glucose level and increases in insulin level in a dose-dependent manner and protective and insulinotrophic action of thymoquinone on pancreatic si-cells was noticed at higher dose.

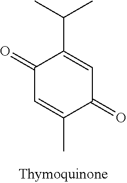

Thymoquinone has been reported to have properties of slow absorption and rapid elimination when administered perorally (Alkharfy K. M. et al, 2015). A nanoformulation of thymoquinone can increase its absorption and bioavailability utilizing natural polymers like chitosan, starch, dextran, carboxymethyl cellulose, albumin, gelatin, alginate, and gums have been widely used. Anionic polymers like gum arabic, gum tragacanth, guar gum, gellan gum and xanthan gum have been also extensively used (Rani R. et al, 2018).

Chloroquine via Gβγ-PLC-IP3-IP3R induces Ca2+ elevation, which in turn promotes GLUT4 fusion with the PM. Moreover, chloroquine can enhance GLUT4 trafficking to the PM. These mechanisms eventually result in glucose uptake increase in control and insulin-resistant L6 cells. These findings suggest that chloroquine might be a potential drug for improving insulin tolerance in diabetic patients (Zhou Q. et al, 2016).

### Definitions

The terminologies used herein describe particular embodiments only and are not intended to be limited. As used in the specification and the appended claims, the singular forms “a,” “and” and “the” include plural references unless the context clearly dictates otherwise.

“About” as used herein may refer to approximately a +/−10% variation from the stated value. It is to be understood that such a variation is always included in any given value provided herein, whether specific reference is made to it.

“Pharmaceutically acceptable medium” means a medium that is compatible with the skin, mucous membranes and the integuments.

A “penetration enhancer” is an agent known to accelerate the delivery of the drug through the skin. These agents also have been referred to as accelerants, adjuvants, and absorption promoters, and are collectively referred to herein as “enhancers.”

The term “fatty acid” means a fatty acid that has four (4) to twenty-four (24) carbon atoms.

The term “supracutaneous administration” means transdermal administration, topical administration, or any combination of the composition

“Pharmacologically effective amount” means that the concentration of the drug is such that in the composition it results in a therapeutic level of drug delivered over the term that the gel is to be used. Such delivery is dependent on a number of variables including the drug, the form of drug, the time period for which the individual dosage unit is to be used, the flux rate of the drug from the gel, surface area of application site, etc. The amount of drug necessary can be experimentally determined based on the flux rate of the drug through the gel, and through the skin when used with and without enhancers.

As used herein, the phrase “in combination with” means that the additional therapeutic agent(s) is administered before, after, or concurrent with the pro-inflammatory cytokine inhibitors, or antigen-binding portion thereof.

The phrase “pharmaceutically acceptable” refers to those supplements, components, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt-, (6) gelatin. (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyloleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations

The term “pharmaceutically-acceptable salts” refers to the relatively non-toxic, inorganic and organic acid addition salts of components of compositions of the present invention. The phrases “systemic administration,” “administered systemically,” “peripheral administration” and “administered peripherally” mean the administration of a subject supplement, composition, therapeutic or other material other than directly into the central nervous system, such that it enters the patient's system and, thus, is subject to metabolism and other like processes, for example, subcutaneous administration.

“Fixed combination” should be understood as meaning a combination whose active principles are combined at fixed doses in the same vehicle/medium (single formula) that delivers them together to the point of application.

“Treatment” as used herein refers to any treatment of a human condition or disease and includes: (1) preventing the disease or condition from occurring in a subject which may be predisposed to the disease but has not yet been diagnosed as having it, (2) inhibiting the disease or condition, 1.e., arresting its development, (3) relieving the disease or condition, i.e., causing regression of the condition, or (4) relieving the conditions caused by the disease, i.e., stopping the symptoms of the disease. The term “bioavailable” means that a compound, composition, supplement, component, or material is in a form that allows for it, or a portion of the amount administered, to be absorbed by, incorporated to, or otherwise physiologically available to a subject to whom it is administered. In certain embodiments of the present invention, bioavailable sources of components of supplements or compositions of the present invention containing a transition metal, including chromium, vanadium, are contemplated, as discussed in more detail herein.

The phrases “parenteral administration” and “administered parenterally” means modes of administration other than enteral and topical administration, usually by injection, and includes, without limitation, intravenous, intramuscular, intra artenial, intrathecal, intracapsular, intra orbital, intracardiac, intradennal, intraperitoneal, transtracheal, subcutaneous, subcuticular, intra-articulare, subcapsular, subarachnoid, intraspinal and intra sterrial injection and infusion.

### BRIEF SUMMARY OF THE INVENTION

The present invention is a method for increasing intracellular glucose uptake to provide energy. The method comprises the administration of a chromium complex in combination with pyridine-3-carboxamide, 2-amino-5-(diaminomethylidene amino) pentanoic acid, (2S)-2-amino-5-(carbamoylamino) pentanoic acid, and/or 2-hydroxybenzoic acid to increase membrane fluidity, while increasing absorption of trace minerals and reducing niconitic acid-induced flushing effects.

Chromium complex and niconitic acid or niacinamide is in a ratio of between about 1:10 (w/w). The composition further comprises at least one of an amino acid such as 2-amino-5-(diaminomethylidene amino) pentanoic acid for vasodilation to increase absorption of chromium complex and to reduce niconitic acid-induced skin flushing effect.

In one aspect, the chromium complex is extended release of chromium in combination with niacin or niacinamide, L-arginine, and/or L-citrulline. Advantageously, the effective amount of niacin is between about 50 and about 10,000 micrograms, and L-arginine is between about 3 and about 10 grams.

In one aspect, the chromium complex includes but not limited to chromium picolinate, chromium chloride, and chromium tripicolinate.

In one aspect, the chromic complex, pyridine-3-carboxamide, and 2-amino-5-(diaminomethylidene amino) pentanoic acid may be incorporated into a tablet, aqueous or oil suspension, dispersible powder or granule, emulsion, hard or soft capsule, syrup or elixir.

In a different aspect, the present invention may also contemplated by this present invention that the composition contains herb such as Nigella sativa. The hexane extract of the herb contains both long and medium chain fatty acids, and therefore exhibits significant enhancement of intestinal absorption for low permeable chromium.

It is also contemplated by this present invention that the composition contains herb extracts such as alpha pinene and boswellic acid. These extracts of the herb contain both long and medium chain fatty acids, and therefore exhibits significant enhancement of intestinal absorption for low permeable chromium.

In one aspect, the components of the composition may also be administered separately.

Compositions may be prepared in according any method known in the art for the manufacture of pharmaceutically acceptable compositions and such compositions may contain one or more of the following agents: sweeteners, flavoring agents, coloring agents and preservatives. Tablets containing the active ingredients in admixture with non-toxic.

In one aspect, dispersible powders and granules of the invention suitable for preparation of an aqueous suspension by the addition of water provide the active ingredient in admixture with a dispersing or wetting agent, a suspending agent, and one or more preservatives. Additional excipients, for example sweetening, flavoring and coloring agents, may also be present.

Although the invention has been described with respect to specific embodiments and examples, it should be appreciated that other embodiments utilizing the concept of the present invention are possible without departing from the scope of the invention. The present invention is defined by the claimed elements, and any modifications, variations, or equivalents that fall within the true spirit and scope of the underlying principles.

### Methods and Uses of the Invention

Administration and Dosages

The compositions are administered in any suitable manner, often with pharmaceutically acceptable carriers. Suitable methods of administering compositions in the context of the present invention to a subject are available, and, although more than one route can be used to administer a particular composition, a particular route can often provide a more immediate and more effective reaction than another route.

The dose administered to a patient, in the context of the present invention, should be sufficient to affect a beneficial therapeutic response in the patient over time, and therefore to inhibit disease progression. Thus, the composition is administered to a subject in an amount sufficient to elicit an effective response to the specific antigens and/or to alleviate, reduce, cure or at least partially arrest symptoms and/or complications from the disease. An amount adequate to accomplish this is defined as a “therapeutically effective dose.”

One skilled in the art will appreciate that the constituents of this formulation may be varied in amounts yet continue to be within the spirit and scope of the present invention. For example, the composition may contain per 100 g of the composition about 100 μg to about 1,500 μg of chromium complex.

Toxicity and therapeutic efficacy of the active ingredients can be determined by standard pharmaceutical procedures, e.g., for determining LD5, (the dose lethal to 50% of the population) and the ED, (the dose therapeutically effective in 50% of the population). The dose ratio between toxic and therapeutic effects is the therapeutic index and it can be expressed as the ratio LD/ED. Compounds which exhibit large therapeutic indices are preferred. While compounds that exhibit toxic effects may be used, care should be taken to design a delivery system that targets such compounds to the site of affected tissue in order to minimize potential damage to uninfected cells and, thereby, reduce side effects.

The present invention is further illustrated by the following examples, which should not be construed as limiting in any way. The contents of all cited references throughout this application are hereby expressly incorporated by reference. The practice of the present invention will employ, unless otherwise indicated, conventional techniques of pharmacology and pharmaceutics, which are within the skill of the art.

### EXAMPLES

The following examples are presented to illustrate presently preferred practice thereof. As illustrations they are not intended to limit the scope of the invention. All quantities are in weight %.

### Example 1

The CrCl3.6H2O (1 mmol, 0.267 g) in 10 mL methanol was mixed with 30 mL methanol of (1.0 mmol, 0.122 g) niacinamide. The mixture was refluxed with stirring at 50° C. for 45 min. The solid chromium (III) complex was isolated after left to be precipitated within one day. This complex was washed with (C2H5)2O ether and then dried over anhydrous CaCl₂).

The effect of chromium compounds on membrane fluidity was assayed with homogeneous liposomes prepared as described by Barenholz et al. (1977). The liposome preparations contained 0.5 mM phosphatidylcholinedioleoyly and 5.0 μM metal complex (chromium picolinate, chromium nicotinate, or zinc picolinate) in 3.0 ml of 50 mM KCl. A preparation that contained 5.0 μM chromium chloride and a control that contained no metal compound (3 ml of 50 mM KCl only) were also prepared. The liposomes were tagged with 1,3-diphenyl-1,3,5-hexatriene (DPH) in tetrahydrofuran. Fluorescence depolarization was determined as a function of temperature with a Jansco spectrofluorometer adapted by connecting the cuvette holder to a circulating water bath.

Also, two polarizers were added to the spectrofluorometer, one in the excitation beam and one in the emission beam. Urine sample assayed using an excitation wavelength of 382 nm and an emission wavelength of 430 nm after an equilibration period of 10 min at each temperature. Emission values were measured with the polarizers both parallel and perpendicular. Corrections for light scattering were made by use of liposomes prepare without DPH. Anisotropy ®, the measure of the bilayer fluidity was calculated by the method of Surkusk et al (1976) as modified by Shinitzky et al (1971).

The result showed that membrane fluidity increased with increasing temperature but the increase in fluidity was greatest when either chromium picolinate or chromium nicotinate, pyridine carboxylate isomer complexes, was added to the medium used for preparation of the liposomes. The increase in fluidity produced by chromium picolinate was much greater than that produced by chromium nicotinate; chromium picolinate is several times more soluble in chloroform (lipophilic) than the nicotinate complex. Thus, the lipophilic complexes used in these experiments produced the most dramatic alterations in membrane fluidity and the most lipophilic of these, chromium picolinate, produced the largest increase in membrane fluidity and also resulted in a marked increase in insulin internalization in cultured cells.

### REFERENCES CITED IN EXAMPLE 1

Syed S, Michalski E S, Tangpricha V, Chesdachai S, Kumar A, Prince J, Ziegler T R, Suchdev P S, Kugathasan S. (2017). Vitamin D status is associated with hepcidin and hemoglobin concentrations in children with inflammatory bowel disease. Inflammation Bowel Discovery 23(9):1650-1658.
Zughaier S M, Alvarez J A, Sloan J H, Konrad R J, Tangpricha V. (2014). The role of vitamin D in regulating the iron-hepcidin-ferroportin axis in monocytes. J Clinical Translational Endocrinol. 1(1):19-25.
Y. Barenholz, D. Gibbes, B. J. Litman, J. Goll, T. E. Thompson, Carlson F. D. (1977). Biochemistry. 16, 2806.
Suurkuusk J., Lentz B. R., Barenholz Y., R. L. Biltonen R. L., and Thompson T. E. (1976). Biochemitry. 15, 1393.
M. Shin&&y, A. C. Dianouz, C. Gilter, and G. Weber, Biochemistry 10, 2106 (1971).

### Example 2

A single oral dose of glucose (2 g/kg bw) was orally administered to each animal and thereafter blood glucose was measured at 0 (just before glucose ingestion), 30 and 60 min after the glucose ingestion using a portable glucometer (Glucoplus Inc., Saint-Laurent, Quebec, Canada).

The result showed that although erythritol did not significantly influence the glucose tolerance ability of normal animals, it significantly improved (p<0.05) the glucose tolerance ability of diabetic animals, especially at 30 and 60 min after the glucose ingestion. On the other hand, erythritol did not significantly influence serum insulin levels in normal animals, but it appreciably increased (p=0.178) diabetes-induced serum insulin depletion of diabetic animals.

### References Cited in Example 2

Chika Ifeany Chukwuma, Ramgopal Mopuri, Savania Nagiah, Anil Amichund Chuturgoon, Md. Shahidul Islam (2018). Erythritol reduces small intestinal glucose absorption, increases muscle glucose uptake, improves glucose metabolic enzymes activities and increases expression of Glut-4 and IRS-1 in type 2 diabetic rats. Eur J Nutr 57:2431-2444.

Therefore, those skilled in the art will appreciate that the conceptions and specific embodiments disclosed in this description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

Throughout this application various publications are referenced. The disclosures of these publications in their entireties are hereby incorporated by reference into this application in order to describe more fully the state of the art to which this invention pertains.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above described exemplary embodiments, but should be defined only in accordance with the following claims appended hereto and their equivalents.

### References

Al-Ali, A., Alkawajah, A., Randhawa, M A., Shaikh, NA. (2008). Oral and intraperitoneal LD50 of thymoquinone, an active principle of Nigella sativa in mice and rats. J Ayub Medicine College Abbottabad, 20:23-27.
Bourgou, S., Ksouri, R., Bellila. A., Skandrani. FH, Marzouk, B. (2008). Phenolic composition and biological activities of Tunisia Nigella Sativa L. shoots and roots. C R Biol, 331:48-55.
Badary, OA., Al-Shabana, OA., Nagi, M N., Al Bekairi, A M. (1998). Acute and subchronic toxicity of thymoquinone in mice. Drug Development Resources, 44:56-61.
Ibrahim, R. M., Hamdan, N. S., Mahmud, R., Imam, M. U., Saini, S. M., Rashid, S. N., Abd Ghafar S. A., Latiff, L. A., Ismail, M. (2014). A randomised controlled trial on hypolipidemic effects of Nigella sativa seeds powder in menopausal women. Journal of Translational Medicine, 12:82.
Najmi, A., Nasiruddin, M., Khan, R. A., Haque, S. F. (2008). Effect of Nigella sativa oil on various clinical and biochemical parameters of insulin resistance syndrome. International Journal Diabetes Development Countries, 28:11-14.
El-Mahmoudy, A., Shimizu, Y., Shiina, T., Matsuyama, H., El-Sayed, M., Takewaki, T. (2005) Successful abrogation by thymoquinone against induction of diabetes mellitus with streptozotocin via nitric oxide inhibitory mechanism. International Immunopharmacology, 5:195-207.
Farah, K. M., Atoji, Y., Shimizu, Y., Shiina, T., Nikami, H., Takewaki, T. (2004). Nigella sativa L. oil in streptozotocin-induced diabetic hamsters. Mechanisms of the hypoglycaemic and immune potentiating effects. Res Vet Sci, 77:123-129
Vincent, JB. (2017). New evidence against chromium as an essential trace element. Journal Nutrition, 147:2212-19.
Salem, M. L. (2005). Immunomodulatory and therapeutic properties of the Nigella Sativa L. Seed. International Immunopharmacology, 5:1749-1770.
Ahmad, A., Husain, A., Mujeeb, M., Khan, S. A., Najmi, A. K., Siddique, N. A., Damanhouri, Z. A., Anwar, F. (2013). A review on therapeutic potential of Nigella sativa: a miracle herb. Asian Pac. J. Trop. Biomed, 3 (5): 337-352.
Anderson R A, Kozlovsky, A. S. (1985). Chromium intake, absorption and excretion of subjects consuming self-selected diets. Am. J Clin Nutr., 41(6):1177-83.
Kubota T, Imaizumi T, Oyama J, et al. (1997). L-arginine increases exercise-induced vasodilation of the forearm in patients with heart failure. Jpn Circ J, 61:471-480.
Chen J., Cui X., Zacharek A., et al. (2007). Niaspan increases angiogenesis and improves functional recovery after stroke. Ann Neurol, 62:49-58.
Penberthy, W. T., (2007). The Niacin flush pathway in recovery from Schizophrenia and how Arginine and Glutamine may provide added benefit. JOM, 27:35-36.
Fararh K, Atoji Y, Shimizu Y, Shiina T, Nikami H, Takewaki T. (2004). Mechanisms of the hypoglycaemic and immunopotentiating effects of Nigella sativa L. oil in streptozotocin-induced diabetic hamsters. Res Vet Sci., 77(2):123-129.
Benhaddou-Andaloussi A, Martineau L C, Spoor D, et al (2008). Antidiabetic activity of Nigella sativa. Seed extract in cultured pancreatic β-cells, skeletal muscle cells, and adipocytes. Pharm Biol., 46(1-2):96-104.
Alimohammadi S, Hobbenaghi R, Javanbakht J, et al (2013). Protective and antidiabetic effects of extract from Nigella sativa on blood glucose concentrations against streptozotocin (STZ)-induced diabetic in rats: an experimental study with histopathological evaluation. Diagn Pathol., 8(1):818.
Kanter M, Meral I, Yener Z, Ozbek H, Demir H (2003). Partial regeneration/proliferation of the B-cells in the Islets of Langerhans by Nigella sativa L. in streptozotocin-induced diabetic rats. Tohoku J Exp Med., 201(4):213-219.
Daryabeygi-Khotbehsaraa R., Golzaranda M., Payam Ghaffarib M., Kurosh Djafarian K. (2017). Nigella sativa improves glucose homeostasis and serum lipids in type 2 diabetes: A systematic review and meta-analysis. Complementary Therapies in Medicine, 35:6-13.
Pari L, Sankaranarayanan C. (2009). Beneficial effects of thymoquinone on hepatic key enzymes in streptozotocin-nicotinamide induced diabetic rats. Life Sci., 85(23):830-834.
Alkharfy K. M., Ahmad A., Khan R. M., Al-Shagha W. M. (2015). Pharmacokinetic plasma behaviors of intravenous and oral bioavailability of thymoquinone in a rabbit model, Eur. J. Drug Metab. Pharmacokinet, 40 (3) 319-323.
Rania R. et al (2018). Improvement of antihyperglycemic activity of nano-thymoquinone in rat model of type-2 diabetes. Chemico-Viological Interactions, 295:119-112.
Zebrowska A, Wo_zniacka A, Juczyn_ska K, et al (2017). Correlation between IL36a and IL17 and activity of the disease in selected autoimmune blistering diseases. Mediators Inflammation, 1-10. https://doi.org/10.1155/2017/8980534.
Kistowska M, Meier B, Proust T, et al (2015). Propionibacterium acnes promotes Th17 and Th17/Th1 responses in acne patients. Journal Investment Dermatology, 135: 110-118.
Xiao S, Jin H, Korn T, et al (2008). Retinoic acid increases Foxp3+ regulatory T cells and inhibits development of Th17 cells by enhancing TGF-beta-driven Smad3 signaling and inhibiting IL-6 and IL-23 receptor expression. Journal of Immunology, 181: 2277-2284.
Hentze M W, Muckenthaler M U, Galy B, Camaschella C. (2010). Two to tango: regulation of Mammalian iron metabolism. Cellular, 142(1):24-38.
Papanikolaou G, Tzilianos M, Christakis J I, et al. (2005). Hepcidin in iron overload disorders. Blood, 105(10):4103-4105.
Kremastinos D T, Farmakis D. (2011). Iron overload cardiomyopathy in clinical practice. Circulation, 124(20):2253-2263.
Deugnier Y, Turlin B. (2007). Pathology of hepatic iron overload. World J Gastroenterol, 13(35):4755-4760.
Rajpathak S N, Crandall J P, Wylie-Rosett J, Kabat G C, Rohan T E, Hu F B. (2009). The role of iron in type 2 diabetes in humans. Biochim Biophys Acta., 1790(7):671-681.
Baudrand R, Gupta N, Garza A E, et al. (2016). Caveolin 1 Modulates Aldosterone-Mediated Pathways of Glucose and Lipid Homeostasis. J Am Heart Association, 5(10):e003845.
Kerkela R, Grazette L, Yacobi R, et al. (2001). Cardiotoxicity of the cancer therapeutic agent imatinib mesylate. Nat Med. 2006; 12(8):908-916. 42. van Oosterom A T, Judson I, Verweij J, et al. Safety and efficacy of imatinib (STI571) in metastatic gastrointestinal stromal tumours: a phase I study. Lancet, 358(9291):1421-1423.
Cumming D C, Yang J C, Rebar R W, Yen S S. (1982). Treatment of hirsutism with spironolactone. JAMA, 247(9):1295-1298.
Ouzan J, Perault C, Lincoff A M, Carre E, Mertes M. (2002). The role of spironolactone in the treatment of patients with refractory hypertension. Am J Hypertens, 15(4 Pt 1):333-339.
Corvol P, Michaud A, Menard J, Freifeld M Mahoudeau J. (1975). Antiandrogenic effect of spirolactones: mechanism of action. Endocrinology, 97(1):52-58.
Palloshi A., Fragasso G. F., Monti L D., Setola E., Valsecchi G et al. (2004). Effect of oral L-Arginine on blood pressure and symptoms and endothelial function in patients with systematic hypertension, positive exercise tests, and normal coronary arteries. Am J Cardiol, 93(7):933-5.
Palmer R M J, Ashton D S, Moncada S. (1988). Vascular endothelial cells synthesize nitric oxide from L-arginine. Nature, 333: 664-6.
Newsholme P, Homem De Bittencourt P I, O'Hagan C, De Vito G, Murphy C, Krause M S. (2010). Exercise and possible molecular mechanisms of protection from vascular disease and diabetes: the central role of ROS and nitric oxide. Clin Sci (Lond), 118(5):341-9.
Iyengar R, Stuehr D J, Marietta M A. (1987). Macrophage synthesis of nitrite, nitrate, and N-nitrosamines: precursors and role of the respiratory burst. Proc Natl Acad Sci USA, 84:6369-73.
Forstermann U, Closs E I, Pollock J S, Nakane M, Schwarz P, Gath I, et al. (1994). Nitric oxide synthase isozymes. Characterization, purification, molecular cloning, and functions. Hypertension, 23:1121-31.
Wascher T C, Graier W F, Dittrich P, Hussain M A, Bahadori B, Wallner S, et al. (1997). Effects of low dose L-arginine on insulin-mediated vasodilatation and insulin sensitivity. Eur J Clin Investment, 27:690-5.
M. Morita, M. Sakurada, F. Watanabe, et al. (2013). Effects of oral L-citrullinesupplementation on lipoprotein oxidation and endothelial dysfunction in humans with vasospastic angina, Immunol. Endocr. Metab. Agents Med. Chemistry, 13:214-220.
N. Kameda, T. Okigawa, T. Kimura, et al. (2011). The effect of L-citrulline ingestion on ECG QT interval and autonomic nervous system activity, J. Physiol. Anthropology, 30: 41-45.
M. J. Romero, D. H. Platt, R. B. Caldwell, et al. (2006). Therapeutic use of citrulline in cardiovascular disease, Cardiovasc. Drug Rev, 24:275-290.
S. Pollock, U. Forstermann, J. A. Mitchell, et al. (1991). Purification and characterization of particulate endothelium-derived relaxing factor synthase from cultured and native bovine aortic endothelial cells, Proc. Natl. Acad. Sci. U.S.A., 88:10480-10484.
S. Shin, S. Mohan, H. L. Fung (2011). Intracellular L-arginine concentration does not determine NO production in endothelial cells: implications on the “L-arginine paradox”, Biochem. Biophys. Res. Community, 414:660-663.
Ahlborg G, Bjorkman O. (1900). Carbohydrate utilization by exercising muscle following pre-exercise glucose ingestion. Clin Phys 7: 181-195.
Ahlborg G, Felig P, Hagenfeldt L, Hendler R, Wahren J. (1974). Substrate turnover during prolonged exercise in man. Splanchinc and leg metabolism of glucose, free fatty acids and amino acids. J Clin Invest 53: 1080-1090.
Antonescu C N, Thong F S L, Niu W, Karnieli E, Klip A. (2011). To be or not to be: regulation of the intrinsic activity of GLUT4. Curr Med Chem Immun Endoc Metab Agents 5: 175-187.
Felig P, Cherif A, Minagawa A, Wahren J. (1982). Hypoglycemia during prolonged exercise in normal men. N Engl J Med 306: 895-900.
Goodyear L J, Hirshman M F, Napoli R, Calles J, Markuns J F, Ljungqvist O, Horton E S. (1996). Glucose ingestion causes GLUT4 translocation in human skeletal muscle. Diabetes 45: 1051-1056.
Greiwe J S, Hickner R C, Hansen P A, Racette S B, Chen M M, Holloszy J O. (1999). Effects of endurance exercise training on muscle glycogen accumulation in humans. J Appl Physiol 87: 222-226.
Guma A, Zierath J R, Wallberg-Henriksson H, Klip A. Insulin induces translocation of GLUT-4 glucose transporters in human skeletal muscle. Am J Physiol Endocrinol Metab 268: E613-E622, 1995.
Holloszy J O, Narahara H T. (1965). Studies of tissue permeability. J Biol Chem 240: 3493-3500.
Holloszy J O, Narahara H T. (1967). Nitrate ions: potentiation of increased permeability to sugar associated with muscle contractions. Science 155: 573-575.
Henriksen E J, Bourey R E, Rodnick K J, Koranyi L, Permutt M A, Holloszy J O. (1990). Glucose transporter protein content and glucose transport capacity in rat skeletal muscles. Am J Physiol Endocrinol Metab 259: E593-E598.
Jeukendrup A E, Raben A, Gijsen A, Stegen J H, Brouns F, Saris W H, Wagenmakers A J. (1990). Glucose kinetics during prolonged exercise in highly trained human subjects: effect of glucose ingestion. J Physiol 515: 579-589.
MacLean D A, Bangsbo J, Saltin B. (1999). Muscle interstitial glucose and lactate levels during dynamic exercise in humans determined by microdialysis. J Appl Physiol 87: 1483-1490.
McConell G, Fabris S, Proietto J, Hargreaves M. (1994). Effect of carbohydrate ingestion on glucose kinetics during exercise. J Appl Physiol 77: 1537-1541.
McCoy M, Proietto J, Hargreaves M. (1996). Skeletal muscle GLUT-4 and postexercise mus cleglycogen storage in humans. J Appl Physiol 80: 411-415.
Zaid H, Talior-Volodarsky I, Antonescu C, Liu Z, Klip A. (2009). GAPDH binds GLUT4 reciprocally to hexokinase-II and regulates glucose transport activity. Biochem J 419: 475-484.
Dugani, C. B. & Klip, A. (2005). Glucose transporter 4: cycling, compartments and controversies. EMBO Rep 6, 1137-1142.
Qi Zhoua Xinzhou Yangb Mingrui Xionga Xiaolan Xuc Li Zhenc Weiwei Chena Yan Wanga Jinhua Shena Ping Zhaoa Qing-Hua L. (2016). Chloroquine Increases Glucose Uptake via Enhancing GLUT4 Translocation and Fusion with the Plasma Membrane in L6 Cells. Cellular Physiology and Biochemistry, 38: 2030-2040.

We claim:

1. A method for increasing glucose uptake in muscle cell to boost energy comprising administering an effect amount of a composition comprising:
an effective amount of at least one GLUT-4 expression inducement agent in combination with an effective amount of antioxidant in combination with at least one amino acid and an effective amount vasodilator, wherein said composition contains said at least one GLUT-4 expression inducement agent in combination with said antioxidant in combination with said at least one amino acid and said vasodilator in an amount and at a ratio therapeutically effective for at least one time per day dosage.

2. The method of claim 1, said at least one GLUT-4 expression inducement agent is erythritol in about a dosage of 10 g to 50 g, preferably 10 g to 20 g, preferably 20 g to 30 g, preferably 30 g to 40 g, preferably 40 g to 50 g.

3. The method of claim 1, wherein said antioxidant is selected from the group consisting of chromium ionic, alpha pinene, caffeine, and any combination thereof.

4. The method of claim 3, wherein said chromium ionic is selected from the group consisting of chromium picolinate, chromium nicotinate, chromium glycinate, or other chromium comprising chromium content of between about 100 micrograms to 1,500 micrograms.

5. The method of claim 1, wherein the concentration of antioxidant metabolite, derivative, or analogue thereof is from 0.01% (w/w) to 1% (w/w).

6. The method of claim 4, wherein said chromium ionic is combined with niacin or niacinamide for increasing absorption rate.

7. The method of claim 1, wherein said amino acid comprises L-arginine in combination with L-citrulline.

8. The method of claim 1, wherein said vasodilator is selected from the group consisting of magnesium glycinate, magnesium stearate, magnesium citrate, magnesium chloride, magnesium oxide, magnesium lactate, magnesium L-threonate, magnesium malate, magnesium orotate, and any combination thereof.

9. The method of claim 1, wherein the concentration of vasodilator and/or vasodilator metabolite, derivative, or analogue thereof is from 1% (w/w) to 15% (w/w).

10. The composition of claim 1 further comprises tocopherol (vitamin E), cholecalciferol (vitamin D3), and frankincense extract.

11. The method of claim 1, wherein in an aqueous environment of said chromium ionic is released into solution rapidly.

12. The method of claim 1 further comprises a pharmaceutically acceptable carrier, adjuvant, excipientor diluent.

13. The method of claim 11, wherein said pharmaceutically acceptable carrier is selected from the group consisting of a pill, capsule, lozenge, caplet, syrup, emulsion, suspensional liquid, powder, spray, cream or lotion.

14. The method of claim 1, wherein said composition is administered orally, nasally, sublingually, intramuscularly, subcutaneously, transdermally, and any combination thereof.
